(19) United States
(12) Patent Application Publication
(10) Pub. No.: US 2025/0009241 A1
(43) Pub. Date: Jan. 9, 2025

(54) SYSTEM AND METHOD FOR MONITORING BLOOD PRESSURE

(71) Applicant: Taipei Medical University, Taipei City (TW)

(72) Inventors: Yu-Chuan Li, Taipei City (TW); Kseniia Sholokhova, Taipei City (TW)

(21) Appl. No.: 18/765,430

(22) Filed: Jul. 8, 2024

Related U.S. Application Data
(60) Provisional application No. 63/525,417, filed on Jul. 7, 2023.

(51) Int. Cl. A61B 5/024; A61B 5/00; A61B 5/021

(52) U.S. Cl. CPC A61B 5/02427; A61B 5/02108; A61B 5/02438; A61B 5/6844; A61B 5/725; A61B 5/7267; A61B 2562/0219

(57) ABSTRACT

Provided is a system and method for monitoring blood pressure, including a mobile device and an apparatus attached to the mobile device. The apparatus includes a PPG device, an accelerometer, a microcontroller, and an external processor for monitoring blood pressure of a subject over a period of time. The attached apparatus utilizes a PPG device to collect the biometric data of the subject while the subject is making physical contact with the apparatus. The cuffless blood pressure monitoring system and method utilize machine learning or deep learning models to estimate and track blood pressure of a subject over a period of time unaware by the subject.

### CROSS-REFERENCE TO RELATED APPLICATION

This application claims the benefit of priority to U.S. Provisional application Ser. No. 63/525,417 filed on Jul. 7, 2023, the entire contents of which are incorporated herein by reference.

### BACKGROUND

### Technical Field

The present disclosure relates to a field of smart blood pressure monitoring. More particularly, the present disclosure relates to a system and method of continuous blood pressure monitoring for a subject over a period of time, utilizing machine learning and/or deep learning techniques.

### Description of Related Art

Blood pressure is one of the most tracked vital signs and is considered as an indicator of cardiovascular diseases. People are advised to keep track of their blood pressures to monitor their overall health status for long term high blood pressure that indicates high risk of stroke, heart diseases such as heart failure and heart attack. It is estimated that 1 in 3 adults has high blood pressure, and 36 millions of adults do not control their high blood pressure well and require continuous monitoring of blood pressure.

However, the gold standard of blood pressure measurement, arterial catheterization, is invasive and only carried out in hospitals. While arterial catheterization provides continuous and accurate measurement of blood pressure, the procedure can lead to severe complications such as hemorrhage, thrombus formation, emboli, distal ischemia, and infection.

On the other hand, cuff-based home monitors for blood pressure measurement are now the most common choice for people. They are non-invasive and safe but only provide a single time point measurement. In addition, people need to take time to sit still and make measurement, which are inconvenient and can interfere with people's daily lives. Very easily, people forget to take measurement in a busy day.

Therefore, there remains an unmet need for a blood pressure monitoring method and system that are safe and convenient such that the measuring process does not interfere with people's busy lives. It is also desirable that the system provides a continuous measurement of blood pressures over time.

### SUMMARY

In view of the foregoing, the present disclosure provides an integrated system with a mobile device and a method that provide continuous measurement of blood pressure in an invisible manner to minimize interruption to people's lives. The present disclosure takes advantage of the widespread use of mobile devices to take blood pressure measurement by integrating the blood pressure measuring apparatus with a mobile device, such that blood pressure is measured whenever a subject holds and makes physical contact with a mobile device, and the subject does not need to wear a wearable device all the time for blood pressure measurement. Not only that the blood pressure measurements can be made several times a day to provide continuous blood pressure measurement over time, the subject would not be aware of when the blood pressure measurements are made. Therefore, the present disclosure provides an effortless system and method for continuous blood pressure monitoring that is convenient and safe.

In at least one embodiment, the present disclosure provides a blood pressure monitoring system comprising a mobile device and an apparatus configured to be attached on the mobile device, wherein the apparatus comprises a photoplethysmography (PPG) device, a microcontroller, an accelerometer, and an external processor. In at least one embodiment of the present disclosure, the PPG device is configured to generate PPG data from one or more sensors. In at least one embodiment of the present disclosure, the sensor is a PPG sensor. In at least one embodiment of the present disclosure, the microcontroller is programmed to collect, process, and store the PPG data. In some embodiments of the present disclosure, the microcontroller is configured to transmit the PPG data to the external processor.

In some embodiments of the present disclosure, the PPG data can be stored locally or transmitted by any protocol or network connection known in the art, wirelessly or wired, such as Bluetooth, Wi-Fi, or Internet-of-Things (IoT) network. In at least one embodiment of the present disclosure, the external processor is configured to estimate a blood pressure from the PPG data by implementing a machine learning model. In at least one embodiment of the present disclosure, the external processor is configured to estimate a blood pressure from the PPG data by implementing a deep learning model. In at least one embodiment of the present disclosure, the deep learning model is a recurrent neural network (RNN), a convolutional neural network (CNN), a transformer model, attention mechanism, a hybrid model, an autoencoder, a generative adversarial network (GAN), a graph neural network (GNN), a WaveNet model, a deep belief network (DBN), a sparse coding, or any combination thereof. The external processor can be a cloud-based server or a physical computer that can communicate through any protocol or network connection known in the art.

In at least one embodiment of the present disclosure, the PPG device, the microcontroller, and the accelerometer are connected on a printed circuit board in the apparatus. In some embodiments, the system of the present disclosure can further comprise a software to visually display estimated blood pressure on the mobile device.

In at least one embodiment of the present disclosure, a method for monitoring blood pressure of a subject is provided by using the system described above. In at least one embodiment, the method comprises: detecting a contact between the subject and the apparatus of the system described above; concurrently recording PPG data and time stamp via the PPG device and the accelerometer, respectively; stop recording the data for a set period of time where a motion of a set level is detected by the accelerometer; transmitting the recorded data to the external processor; and estimating a blood pressure by implementing at least one of a machine learning model and a deep learning model with the data received at the external processor. The level of motion and duration of time for stopping the recording can be determined and adjusted by a skilled person in the art to obtain a proper blood pressure result.

For example, when a subject is holding a mobile device with the blood pressure measuring apparatus attached and the subject starts to climb a flight of stairs, provoking a significant motion, the apparatus stops recording data for 3 to 5 seconds.

In at least one embodiment, the PPG data are recorded from an analog channel. In at least one embodiment, the PPG data are raw signals. In at least one embodiment, the raw PPG signals undergo data preprocessing before being used to train or implement a machine learning model or a deep learning model. In at least one embodiment, the machine learning model is Random Forest regressor or XGBoost regressor. In at least one embodiment, the deep learning model is a recurrent neural network (RNN), a convolutional neural network (CNN), a transformer model, attention mechanism, a hybrid model, an autoencoder, a generative adversarial network (GAN), a graph neural network (GNN), a WaveNet model, a deep belief network (DBN), a sparse coding, or any combination thereof. In at least one embodiment, the machine learning model is trained and tested with a dataset having PPG data and arterial blood pressure (ABP) data from a same heartbeat. In at least one embodiment, the PPG data and the ABP data are preprocessed by filtering and normalization. In at least one embodiment, the PPG data and the ABP data are refined to provide an optimal quality for deep learning model.

In at least one embodiment of the present disclosure, at least one feature is extracted from the waveform contour of the PPG data by the machine learning model, including the distance from the diastolic peak to the systolic peak, the systolic phase, the diastolic phase, the distance from the onset to the tangent intersection point (tip) of signal, the ratio of the diastolic time over the systolic time, and the distance from the diastolic peak to the tangent intersection point (tip). In at least one embodiment of the present disclosure, at least one feature is extracted from the arterial blood pressure data, including systolic blood pressure and diastolic blood pressure. In at least one embodiment, the blood pressure monitoring method further comprises displaying the estimated blood pressure on the mobile device. In at least one embodiment of the present disclosure, the one or more features to be extracted from the PPG data are determined by a deep learning model. In at least one embodiment of the present disclosure, a deep learning model learns and extracts one or more features from the raw PPG data. In at least one embodiment of the present disclosure, a deep learning model learns and extracts one or more features from preprocessed or refined PPG data.

It should be understood that the summary above is provided to introduce, in simplified form, a selection of concepts that are further described in the detailed description below. It is not meant to identify key or essential features of the claimed subject matter, the scope of which is defined by the claims that follow the detailed description. Furthermore, the claimed subject matter is not limited to implementations that solve any disadvantages noted above or in any part of this specification. The above advantages and other advantages and features of the present disclosure will be readily apparent from the following detailed description when taken alone or in combination with the accompanying drawings. The disclosure extends to any combination of features disclosed herein, whether or not such a combination is mentioned explicitly herein. Further, where two or more features are mentioned in combination, for example in the same paragraph, it is intended that such features may be claimed separately without extending the scope of the disclosure. Features from different embodiments described below may also be brought together in any claim.

### BRIEF DESCRIPTION OF DRAWINGS

The present disclosure can be put into effect in numerous ways, illustrative non-limiting embodiments of which are described below with reference to the accompanying drawings.

FIG. 1 shows a schematic framework of an exemplary system in an embodiment of the present disclosure.

FIG. 2 shows a block diagram of estimating blood pressure by machine learning module.

FIGS. 3A and 3B show the diastolic blood pressure (DBP) and systolic blood pressure (SBP) prediction results. In Bland-Altman plots, the dotted line in the middle denotes the mean difference between the actual and estimated blood pressure (BP) values. The dashed lines above and below represent the limits of agreement. In distribution of errors plots, the presented histograms indicate that the errors in DBP and SBP are distributed in a bell-shaped curve centered around zero, suggesting a normal distribution.

FIG. 4 shows a histogram comparing the model performance in capturing variations in DBP and SBP values.

FIG. 5 shows a histogram comparing features importance for prediction of DBP and SBP. dt: distance from the systolic peak to the end of signal; dia_tip: distance from the diastolic peak to the tangent intersection point; delta: distance from the diastolic peak to the systolic peak; ratio_dia_sys: the ratio of diastolic time to systolic time; ct: distance from the beginning of the signal to the peak of systole; onsets: distance from the onset to the tangent intersection point.

### DETAILED DESCRIPTION

The following examples are used for illustrating the present disclosure. A person skilled in the art can easily conceive the other effects of the present disclosure, based on the disclosure of the specification. It will be apparent that one or more embodiments may be practiced without specific details. The present disclosure can also be implemented or applied as described in different examples. It is possible to modify or alter the following examples for carrying out this disclosure without contravening its scope for different applications.

In this disclosure, all terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein are defined based on the meaning of the terms together with the descriptions throughout the specification.

As used in this disclosure, the singular forms “a,” “an,” and “the” include plural referents unless expressly and unequivocally limited to one referent. The term “or” is used interchangeably with the term “and/or” unless the context clearly indicates otherwise.

Also, when a part “includes” or “comprises” a component or a step, unless there is a particular description contrary thereto, the part can further include other components or other steps, not excluding the others.

Notwithstanding that the numerical ranges and parameters setting forth the broad scope of the disclosure are approximations, the numerical values set forth in the examples are reported as precisely as possible. Any numerical value, however, inherently contains certain errors resulting from the standard deviation found in the respective testing measurements. Accordingly, unless indicated to the contrary, the numerical parameters set forth in the present disclosure and attached claims are approximations that can be varied. At the very least, each numerical parameter should at least be construed in light of the number of reported digits and by applying ordinary rounding techniques. All given ranges and values may vary by 1% to 5%, unless indicated otherwise or known otherwise by a person skilled in the art. Therefore, the term “about” is usually omitted from the description and claims.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the term “microcontroller” means a compact integrated circuit that incorporates a central processing unit, a memory, and input/output functions.

As used herein, the term “mobile device” can be any handheld or compact device to be carried by a subject and to which several physical contacts are made in a day, such as a mobile phone or a tablet. For example, the mobile device may be a mobile phone. In some embodiments, the mobile device is not a wearable device such as a smartwatch, a fitness tracker, a smart ring, and smart glasses.

Reference will now be made in detail to the exemplary embodiments of the disclosure, with some of them illustrated in the accompanying drawings.

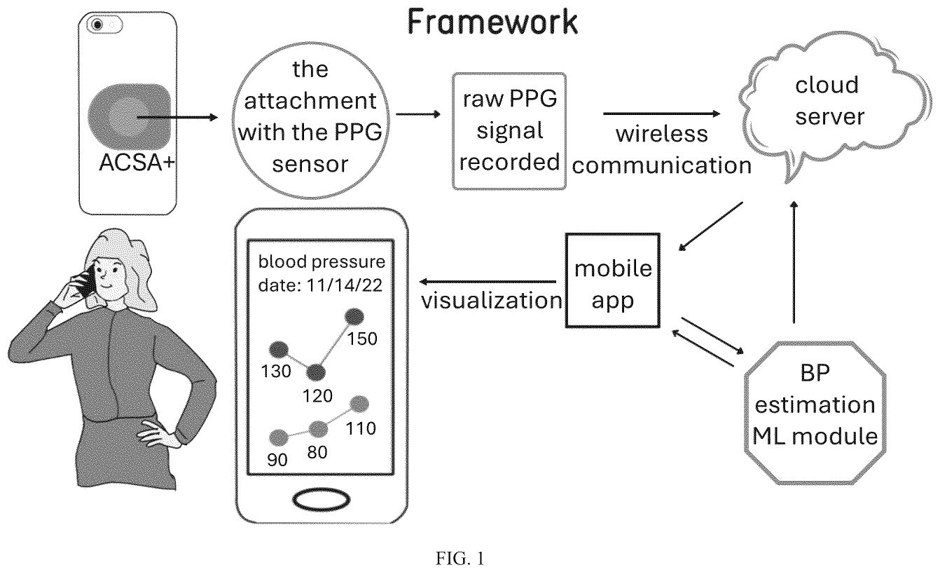

According to an exemplary embodiment of the present disclosure, a scheme of framework of a blood pressure monitoring system is shown in FIG. 1. Referring to FIG. 1, the blood pressure monitoring system comprises: a mobile phone and an attachment on the mobile device, wherein the attachment comprises a photoplethysmography (PPG) device, a microcontroller, an accelerometer, and an external processor. The microcontroller records raw PPG signals when a user is talking on the phone and holding the phone in his/her hand. The recorded PPG signals are then transmitted wirelessly to a cloud server, with the data from the accelerometer also transmitted to the cloud simultaneously with the raw PPG signals. Afterwards, the blood pressure is estimated by a machine learning module implemented by the cloud server. The estimated blood pressure result is transmitted and stored in the cloud server or sent to a mobile application on the phone to be visualized by the user.

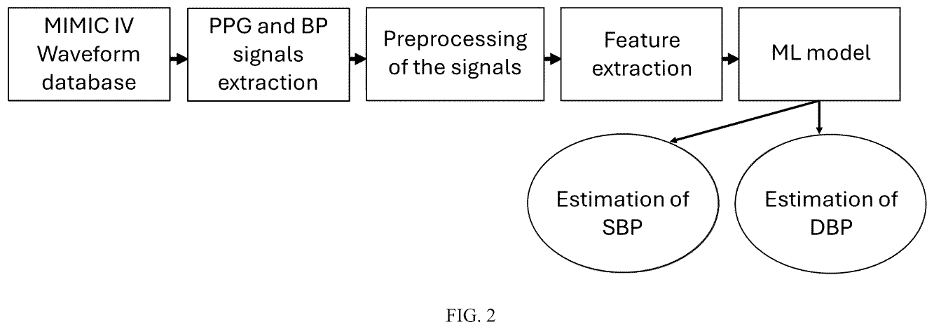

In some embodiments, to estimate blood pressure from the recorded PPG signals, a machine learning (ML) module was carried out as shown in FIG. 2. To begin with, a database containing simultaneous presence of arterial blood pressure (ABP) and PPG signals from the same heartbeat was used, such as the Medical Information Mart for Intensive Care (MIMIC) IV waveform database from Massachusetts Institute of Technology, which is the latest version of MIMIC series database containing multiple vital signs collected from Intensive Care Unit (ICU) patients. From this database, 57 patients satisfied the condition of the simultaneous presence of ABP and PPG signals and acceptable duration time; however, only 9 patients had good and acceptable qualities of both signals, resulting in 922 waveforms. These 922 waveforms were then preprocessed by first synchronizing the signals and then filtered to remove the low and high pass frequencies. Too low and too high blood pressure values were also excluded from analysis. For feature extraction from the ABP and PPG signals, two variables, systolic blood pressure (SBP) and diastolic blood pressure (DBP), were extracted from the ABP signals using the peak detection algorithm, which correspond to the highest value in systole and the lowest value in diastole. From the PPG signals, six features were extracted based on the PPG waveform contour, including the systolic phase, the diastolic phase, the distance from the diastolic peak to the systolic peak, the distance from the onset to the tip of signal, the distance from the tip to the peak of the diastole, and the ratio of the diastolic time over the systolic time. Then, the features from the same heartbeat-generated PPG signals and ABP signals were matched.

To predict BP values from the six features of PPG signals, Random Forest Regression and XGBoost Regression models were implemented. The data were divided into 70% training set and 30% test set. The performances of the prediction power of the two models are shown in Table 1 below.

TABLE 1
Prediction performance of two machine learning model

| ML model | Prediction task | MAE (mmHg)* | SD (mmHg)** |
|---|---|---|---|
| Random Forest | SBP | 4.44 | ±5.14 |
| Regressor | DBP | 2.52 | ±3.16 |
| XGBoost | SBP | 4.54 | ±5.31 |
| Regressor | DBP | 2.74 | ±3.22 |

*MAE: mean absolute error
**SD: standard deviation

To understand the performance of the model, the result can be compared with the AAMI standards for cuffless blood pressure estimation, as illustrated in Table 2.

TABLE 2
Comparison with the AAMI standards for
cuffless blood pressure estimation

| | BP Metric | Mean Error | Standard Deviation |
|---|---|---|---|
| AAMI | SBP | ≤5 (mmHg) | ≤8 (mmHg) |
| Standards | DBP | ≤5 (mmHg) | ≤8 (mmHg) |

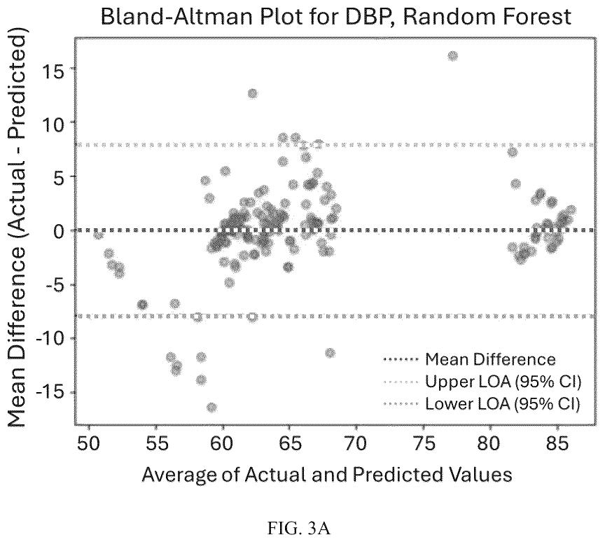

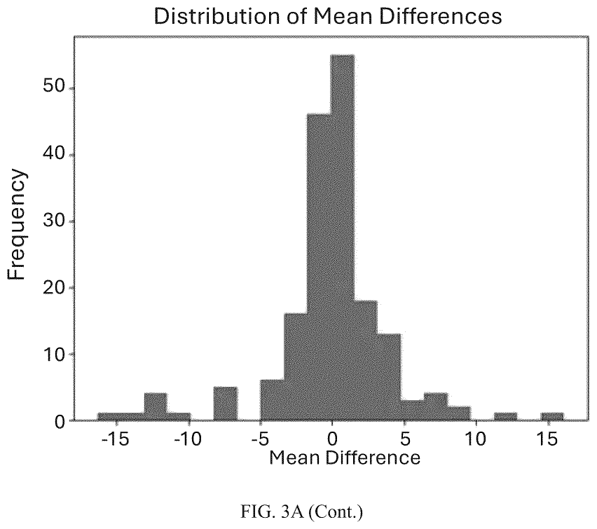

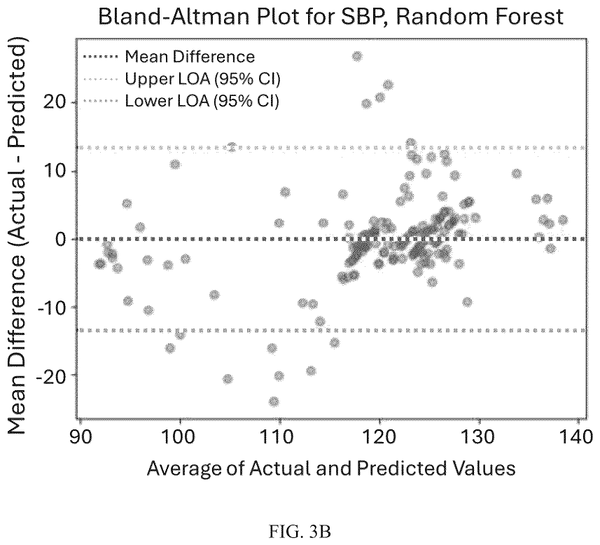

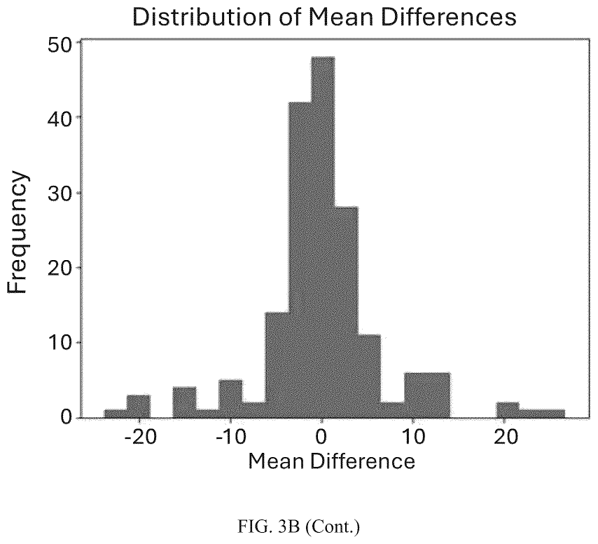

According to the mean error and its standard deviation calculation plotted on Bland-Altman plots, as shown in FIGS. 3A and 3B, both models' DBP and SBP prediction results meet the Standards from the Association for the Advancement of Medical Instrumentation (AAMI), i.e., a mean error (ME) less than 5 (mmHg) and a standard error (SD) less than 8 (mmHg).

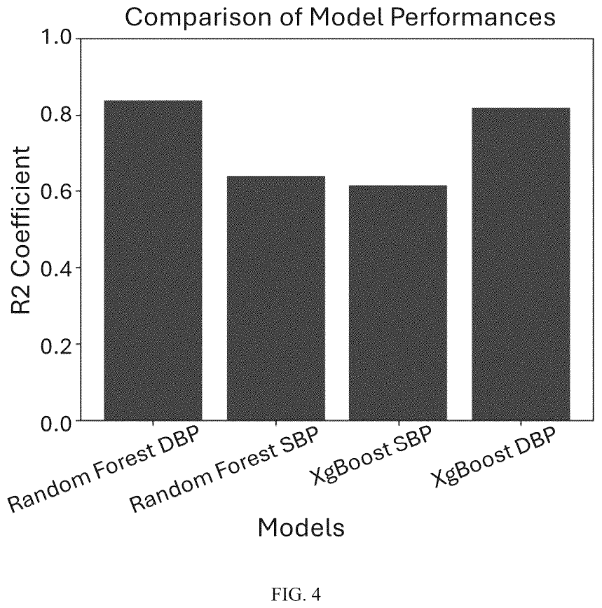

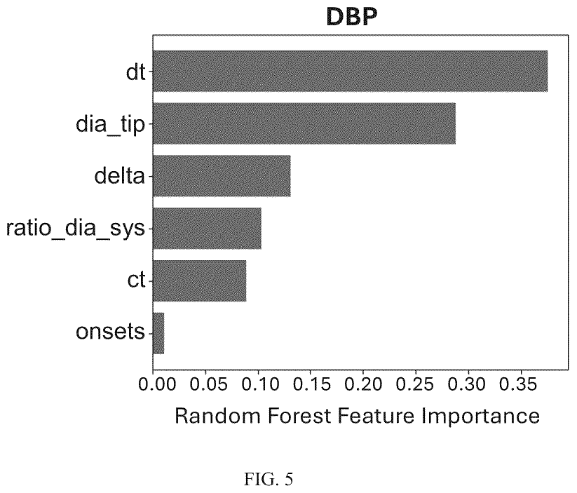

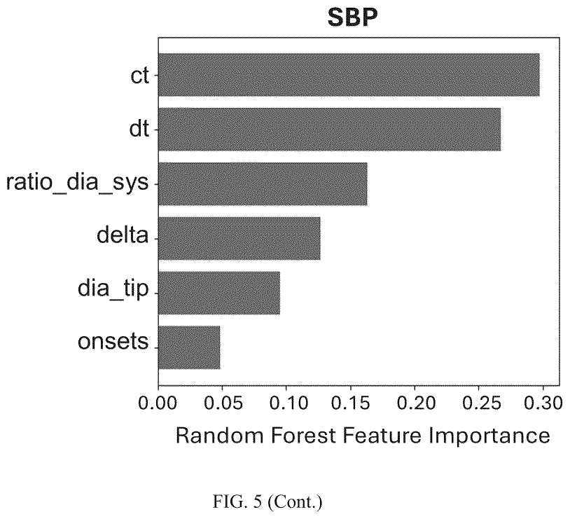

FIG. 4 shows R² coefficients of the two models, and an R² coefficient of 0.8 indicates that the prediction model captures a substantial amount of the variation in DBP and SBP values. Feature importance for prediction of DBP and SBP is analyzed and shown in FIG. 5. It is shown that for predicting DBP, features directly associated with diastolic peak (including distance from the systolic peak to the end of signal (dt) and distance from the diastolic peak to the tangent intersection point (dia_tip)) were more significant than other features. For predicting SBP, the features related to diastole and systole period (including distance from the beginning of the signal to the peak of systole (ct) and distance from the systolic peak to the end of signal (dt)) were comparably significant.

In other embodiments, the deep learning techniques were integrated into existing framework to achieve continuous blood pressure monitoring. This integration involves:
1. Data Preprocessing: Refining the raw PPG signals to ensure optimal input quality for deep learning models;
2. Model Training: Implementing and training deep learning models such as RNNs, GRUs, long short-term memory (LSTM) networks, CNNs, temporal convolutional networks (TCNs) transformers, attention mechanisms, hybrid models (CNN-LSTM, CNN-GRU), autoencoders, GANs, GNNs, DBNs, sparse coding, and WaveNet models on the preprocessed PPG data;
3. Performance Evaluation: Comparing the performance of these deep learning models against traditional methods in terms of prediction accuracy and clinical relevance;
4. Continuous Monitoring: Developing a robust system capable of continuous, real-time blood pressure monitoring, which can provide valuable insights into blood pressure variability and the effectiveness of antihypertensive treatments.

In some embodiments, the deep learning modeling can provide continuous monitoring of blood pressure without the need for deliberate feature extraction, leveraging the capability of these models to automatically learn and extract relevant features directly from the raw data. In some embodiments, the present disclosure provides a more accurate representation of blood pressure throughout different activities and times of the day. In other embodiments, the present disclosure can also assess the effectiveness of antihypertensive medications. In some embodiments, the present disclosure can also prevent an acute hypertensive event by detecting sudden spikes in blood pressure or managing chronic hypertension.

While some of the embodiments of the present disclosure have been described in detail in the above, it is, however, possible for those of ordinary skill in the art to make various modifications and changes to the embodiments shown without substantially departing from the teaching of the present disclosure. Such modifications and changes are encompassed in the scope of the present disclosure as set forth in the appended claims.

What is claimed is:

1. A system for monitoring blood pressure, comprising:
a mobile device; and
an apparatus configured to be attached on the mobile device, wherein the apparatus comprises a photoplethysmography (PPG) device, a microcontroller, an accelerometer, and an external processor.

2. The system of claim 1, wherein the PPG device is configured to generate PPG data from one or more sensors.

3. The system of claim 2, wherein the microcontroller is programmed to collect, process, and store the PPG data.

4. The system of claim 2, wherein the microcontroller is configured to transmit the PPG data to the external processor.

5. The system of claim 2, wherein the external processor is configured to estimate a blood pressure from the PPG data by implementing a machine learning model.

6. The system of claim 2, wherein the external processor is configured to estimate a blood pressure from the PPG data by implementing a deep learning model.

7. The system of claim 1, wherein the PPG device, the microcontroller, and the accelerometer are connected on a printed circuit board.

8. The system of claim 1, further comprising a software to visually display estimated blood pressure on the mobile device.

9. A method for monitoring blood pressure of a subject, comprising:
detecting a contact between the subject and the apparatus of claim 1;
concurrently recording PPG data and time stamp via the PPG device and the accelerometer, respectively;
stop recording the data for a set period of time where a motion of a set level is detected by the accelerometer;
transmitting the recorded data to the external processor; and
estimating a blood pressure by implementing at least one of a machine learning model and a deep learning model at the external processor.

10. The method of claim 9, wherein the machine learning model is Random Forest regressor or XGBoost regressor.

11. The method of claim 9, wherein the machine learning model is trained and tested with a dataset having PPG data and arterial blood pressure (ABP) data from a same heartbeat.

12. The method of claim 11, wherein the PPG data and the ABP data are preprocessed by filtering and normalization.

13. The method of claim 11, wherein at least one feature is extracted from a waveform contour of the PPG data.

14. The method of claim 13, wherein the feature is a systolic phase, a diastolic phase, a distance from a diastolic peak to a systolic peak, a distance from an onset to a tip of signal, a distance from a tip to a peak of a diastole, a ratio of diastolic time over systolic time, or any combination thereof.

15. The method of claim 11, wherein at least one feature is extracted from the arterial blood pressure data.

16. The method of claim 15, wherein the at least one feature is systolic blood pressure or diastolic blood pressure.

17. The method of claim 9, wherein the deep learning model is a recurrent neural network (RNN), a convolutional neural network (CNN), a transformer model, attention mechanism, a hybrid model, an autoencoder, a generative adversarial network (GAN), a graph neural network (GNN), a WaveNet model, a deep belief network (DBN), a sparse coding, or any combination thereof.

18. The method of claim 9, further comprising displaying the estimated blood pressure on the mobile device.

19. The method of claim 9, wherein the PPG data are recorded from an analog channel.

20. The method of claim 9, wherein the PPG data are raw signals.
